(19) United States
(12) Patent Application Publication
(10) Pub. No.: US 2016/0137685 A1
(43) Pub. Date: May 19, 2016

(54) NUCLEOSIDE PHOSPHORAMIDATES AND PHOSPHORAMIDITES

(71) Applicant: Hasmukh B. Patel, Edison, NJ (US)

(72) Inventor: Hasmukh B. Patel, Edison, NJ (US)

(21) Appl. No.: 15/002,377

(22) Filed: Jan. 20, 2016

Related U.S. Application Data
(63) Continuation-in-part of application No. PCT/US14/47721, filed on Jul. 23, 2014.
(60) Provisional application No. 61/858,184, filed on Jul. 25, 2013.
(60) Provisional application No. 61/863,948, filed on Aug. 9, 2013.

(51) Int. Cl. C07H 19/10; C07H 19/04; C07H 15/252

(52) U.S. Cl. CPC C07H 19/10; C07H 15/252; C07H 19/04

(57) ABSTRACT

The invention provides phosphoramidate prodrugs for targeting and treating liver disease, including liver cancer, hepatocellular carcinoma, and hepatitis.

### BACKGROUND OF THE INVENTION

Hepatocellular carcinoma (HCC), the most common type of adult liver cancer, is the third leading cause of cancer deaths worldwide (Block T M et al. (2003), Oncogene 22, pp. 5093-5107). Many patients with HCC remain asymptomatic until the disease is in its advanced stages, resulting in ineffective treatment and poor prognosis; the majority of unresectable HCC patients die within one year. The clinical management of HCC can be expected to improve dramatically with improved screening tools to detect the carcinoma in the early stage. Another form of liver cancer is liver-metastasis (including colorectal liver cancer). This type of cancer, although different in the origin, migrates into liver and leads to symptoms similar to some extent to HCC. The focus of this present invention is on both HCC and liver metastases.

The major risk factors of HCC are chronic infections with hepatitis B or hepatitis C virus (HBV or HCV, respectively). Chronic hepatitis can progress into cirrhosis (a noncancerous liver disease associated with fibrosis and abnormal nodules), which increases the risk of developing HCC. Patients with chronic hepatitis and/or cirrhosis, therefore, form a high risk population which would benefit from regular screening for HCC. Current screening tests for HCC are the measurement of alpha-fetoprotein (AFP) levels in the blood serum and the conduction of a hepatic ultrasound. Elevated serum AFP is, however, not a specific marker for HCC, since it is detected in a wide variety of non-hepatic malignancies and benign conditions, including acute and chronic hepatitis (McIntire K R et al. (1975), Cancer Res. 35, pp. 991-996; Liaw Y F (1986), Liver 6, pp. 133-137). Furthermore, 30-50% of HCC cases do not present with elevated serum AFP (Johnson P J (2001), Clin. Liver Dis. 5, pp. 145-159). As a consequence, the AFP test can miss 50% of the positives due to its lack of sensitivity and specificity. A majority of HCC patients concomitantly suffers from cirrhosis. In those patients, the use of advanced imaging technology such as hepatic ultrasound is difficult and frequently non-conclusive.

In liver cancer (HCC and metastases) chemotherapy treatment is sometimes given by Hepatic Artery Infusion to limit exposure of other organs of the body than liver to the chemotherapeutic agents. This practice is very expensive and can only be performed in the clinic, limiting it to a smaller number of patients.

### SUMMARY OF THE INVENTION

The inventor has invented a novel idea by which chemotherapeutic agents (with phosphoramidate and phosphoramidite chemistry) can be delivered orally, thus eliminating expensive and limiting treatments only provided by clinics. This approach (oral therapy) will make this treatment available to significant larger population of patients worldwide of life saving agents.

The present invention is based on unique property of the phosphoramidate and phosphoramidite chemistry. This unique property is such that it provides a targeting mechanism to liver. Hence use of such chemistry will lead to higher effective concentrations of anticancer agents in liver tissue as against other tissues of the body. Having such selectivity will reduce the toxic side effects of the anticancer agent on other parts of the body thus by increasing Therapeutic Index (TI) of the anticancer agent with respect to liver tissue. Another important factor is first-pass-effect encountered by these agents when administered orally. Because these agents (with phosphoramidate and phosphoramidite chemistry) are first taken up by liver when given orally the concentrations are much higher in liver tissue as compared to other organs. Furthermore, these anticancer agents are immediately metabolized such that their presence is localized preventing exit from liver to systemic circulation.

Thus one of the implied invention by the inventor is the identification of the liver targeting property of phosphoramidate and phosphoramidite chemistry and subsequent use of these chemistries in targeting anticancer drugs to HCC and liver metastases.

It is known in anticancer chemotherapy that the liver cancer drugs, such as FUDR, are given by Hepatic Artery Infusion to localize the effect (increased Therapeutic Index) of the anticancer agent. This requires surgery and implanting a pump to deliver drug to liver over a period of time. Thus it is the present inventor's novel idea that these agents now can be targeted without surgery to liver by giving the same agents orally using phosphoramidate and phosphoramidite chemistry attached to the anti-liver-cancer drugs.

Furthermore, it has also been identified by the present inventor that the phosphoramidate and phosphoramidite chemistries also improves targeting to cancerous tissues with better penetration in tumor.

The present invention provides for the compound of formula (I), (Ic), (II), (IIa), (III), and (IIIa), as illustrated herein. The present invention also provides for a pharmaceutical composition that includes one or more of compounds of formula (I), (Ic), (II), (IIa), (III), and (IIIa) in combination with a pharmaceutically acceptable carrier.

The present invention also provides for a method of treating cancer. The method includes administering an effective amount of a compound of formula (I), (Ic), (II), (IIa), (III), or (IIIa) or an effective amount of the pharmaceutical composition that includes one or more of such compounds, to a subject in need of such treatment, for a period of time effective to treat the cancer.

The present invention also provides for a method of treating a viral disease. The method includes administering an effective amount of a compound of formula (I), (Ic), (II), (IIa), (III), or (IIIa) or an effective amount of the pharmaceutical composition that includes one or more of such compounds, to a subject in need of such treatment, for a period of time effective to treat the viral disease.

The present invention also provides for a method for the palliative management of gastrointestinal adenocarcinoma metastatic to the liver. The method includes administering an effective amount of one or more of compounds of formula (I), (Ic), (II), (IIa), or (III), or an effective amount of the pharmaceutical composition that includes one or more of such compounds, to a subject in need of such treatment, for a period of time effective to provide such palliative management.

The present invention also provides for a method of targeting a tumor or cancer cell. The method includes contacting mammalian tissue with an effective amount of a compound of formula (I), (Ic), (II), (IIa), (III), or (IIIa) or an effective amount of the pharmaceutical composition that includes such one or more of such compounds.

The present invention also provides for a method of diagnosing viral or cancer diseases. The method includes administering an effective amount of a compound of formula (III), or an effective amount of the pharmaceutical composition that includes one or more of such compounds, to a subject in need of such diagnosis, for a period of time effective for diagnosis.

One embodiment of the invention provides a method of treating liver disease selected from liver cancer and hepatitis, the method comprising:

administering an effective amount of a compound of formula (III):

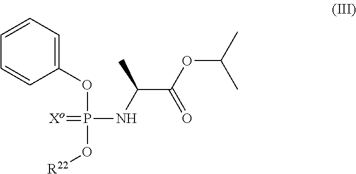

or a pharmaceutically acceptable thereof, wherein, R²² is a monovalent substituent group of a chemotherapeutic agent, or is a monovalent substituent group of an antiviral agent; and X^{O} is O or absent; to a subject in need of such treatment, in a dosage and for a time effective to treat the liver disease; wherein the compound of formula (III) is not sofosbuvir or PSI-7851.

### BRIEF DESCRIPTION OF THE DRAWINGS

FIG. 1. Floxphosteridine and floxuridine anticancer activity in vitro against SK-HEP-1 hepatic cancer cells. SK-HEP-1 hepatic cancer cells were treated with various concentrations (0.01 μM-20 μM) of floxphosteridine and floxuridine for 72 hours, and cell viability was evaluated with MTT cell viability assay.

FIG. 2. Floxphosteridine and floxuridine anticancer activity in vitro against HEPG2 hepatic cancer cells. HEPG2 hepatic cancer cells were treated with various concentrations (1.0 μM-20 μM) of floxphosteridine and floxuridine for 72 hours, and cell viability was evaluated with MTT cell viability assay.

FIG. 3. Floxphosteridine and floxuridine anticancer activity in vitro against AGS gastric cancer cells. AGS gastric cancer cells were treated with various concentrations (0.01 μM-20 μM) of floxphosteridine and floxuridine for 72 hours, and cell viability was evaluated with MTT cell viability assay.

### DETAILED DESCRIPTION OF THE INVENTION

Reference will now be made in detail to certain embodiments of the invention, examples of which are illustrated in part in the accompanying drawings. While the invention will be described in conjunction with the enumerated claims, it will be understood that the exemplified subject matter is not intended to limit the claims to the invention.

First, the term “liver cancer” as used herein is defined to include any cancer located in the liver, including cancers that originate in the liver and those that metastasize to the liver after originating elsewhere in the body. One type of liver cancer is hepatocellular carcinoma (HCC), which originates in the liver.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods of manufacturing described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” can allow for a degree of variability in a value or range, for example, within 10%, within 5%, or within 1% of a stated value or of a stated limit of a range.

The compounds of the invention exclude compounds heretofore known. With respect to the United States, the compounds or compositions herein exclude compounds that are anticipated under 35 U.S.C. §102, and that are obvious under 35 U.S.C. §103.

Whenever a compound described herein is substituted with more than one of the same designated group (e.g., more than one of the same designated variable, substituent group, substituent, etc.), then it will be understood that each occurrence of the designated group may be the same or may be different, i.e., each designated group is “independently” selected. By way of illustration, with regard to the compound of formula (I), reference to “R⁹-R¹¹ are each independently hydrogen or alkyl” is understood to mean that the occurrence of R⁹ is hydrogen or alkyl. Independent of R⁹, R¹⁰ is hydrogen or alkyl. Likewise, independent of R⁹ and R¹⁰, R¹¹ is hydrogen or alkyl. This applies to all other groups (e.g., variables, substituent groups, substituents, etc.).

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The term “halo” refers to fluoro, chloro, bromo, and iodo. Similarly, the term “halogen” refers to fluorine, chlorine, bromine, and iodine.

As used herein, “stable compound” and “stable structure” are meant to indicate a compound that is sufficiently robust to survive isolation to a useful degree of purity from a reaction mixture. Only stable compounds are contemplated by the present invention.

As to any of the above groups, which contain one or more substituents, it is understood, of course, that such groups do not contain any substitution or substitution patterns which are sterically impractical and/or synthetically non-feasible.

The term “nitrogenous base” refers to a nitrogen-containing molecule having the chemical properties of a base. It is an organic compound that owes its property as a base to the lone pair of electrons of a nitrogen atom. In biological sciences, nitrogenous bases are typically classified as the derivatives of two parent compounds, pyrimidine and purine. They are non-polar and due to their aromaticity, planar. Both pyrimidines and purines resemble pyridine and are thus weak bases and relatively unreactive towards electrophilic aromatic substitution. Their flat shape is particularly important when considering their roles in nucleic acids as nucleobases (building blocks of DNA and RNA): adenine, guanine, thymine, cytosine, and uracil. These nitrogenous bases hydrogen bond between opposing DNA strands to form the rungs of the “twisted ladder” or double helix of DNA or a biological catalyst that is found in the nucleotides. Adenine is always paired with thymine, and guanine is always paired with cytosine. Uracil is only present in RNA: replacing thymine and pairing with adenine.

The term “adenine” refers to a nucleobase (a purine derivative) with a variety of roles in biochemistry including cellular respiration, in the form of both the energy-rich adenosine triphosphate (ATP) and the cofactors nicotinamide adenine dinucleotide (NAD) and flavin adenine dinucleotide (FAD), and protein synthesis, as a chemical component of DNA and RNA. The shape of adenine is complementary to either thymine in DNA or uracil in RNA.

The term “guanine” refers to one of the four main nucleobases found in the nucleic acids DNA and RNA, the others being adenine, cytosine, and thymine (uracil in RNA). In DNA, guanine is paired with cytosine. With the formula C₅H₅N₅O, guanine is a derivative of purine, consisting of a fused pyrimidine-imidazole ring system with conjugated double bonds. Being unsaturated, the bicyclic molecule is planar. The guanine nucleoside is called guanosine.

The term “thymidine” refers to a chemical compound, more precisely a pyrimidine deoxynucleoside. Deoxythymidine is the DNA nucleoside T, which pairs with deoxyadenosine (A) in double-stranded DNA. In cell biology it is used to synchronize the cells in S phase.

The term “cytosine” refers to one of the four main bases found in DNA and RNA, along with adenine, guanine, and thymine (uracil in RNA). It is a pyrimidine derivative, with a heterocyclic aromatic ring and two substituents attached (an amine group at position 4 and a keto group at position 2). The nucleoside of cytosine is cytidine. In Watson-Crick base pairing, it forms three hydrogen bonds with guanine.

The term “uracil” refers to one of the four nucleobases in the nucleic acid of RNA.

The term “trifluoromethyl” refers to the group —CF₃.

The term “hydroxyl alkyl” refers to the group HO-alkyl-.

The term “nitro” refers to the group —NO₂.

The term “cyano” refers to the group —CN.

The term “hydrogen” refers to the group —H.

As used herein, “alkyl” refers to refers to a group derived from a straight or branched chain saturated hydrocarbon containing from one to eighteen carbon atoms. “Alkyl” can be C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (1-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. The alkyl groups of the present invention are optionally substituted with one, two, three, four, or five substituents.

As used herein, “aryl” refers to refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present invention can be attached to the parent molecular moiety through any substitutable carbon atom in the group. Representative examples of aryl groups include, but are not limited to, indanyl, indenyl, naphthyl, phenyl, and tetrahydronaphthyl. The aryl groups of the present invention are optionally substituted with one, two, three, four, or five substituents.

As used herein, “heterocycle” or “heterocyclyl” refers to refers to a four-, five-, six-, or seven-membered ring containing one, two, three, or four heteroatoms independently selected from nitrogen, oxygen, and sulfur. The four-membered ring has zero double bonds, the five-membered ring has zero to two double bonds, and the six- and seven-membered rings have zero to three double bonds. The term “heterocyclyl” also includes bicyclic groups in which the heterocyclyl ring is fused to another monocyclic heterocyclyl group, or a four- to six-membered aromatic or non-aromatic carbocyclic ring; as well as bridged bicyclic groups such as 7-azabicyclo[2.2.1]hept-7-yl, 2-azabicyclo[2.2.2]oc-2-tyl, and 2-azabicyclo[2.2.2]oc-3-tyl. The heterocyclyl groups of the present invention can be attached to the parent molecular moiety through any carbon atom or nitrogen atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, thiomorpholinyl, 7-azabicyclo[2.2.1]hept-7-yl, 2-azabicyclo[2.2.2]oc-2-tyl, and 2-azabicyclo[2.2.2]oc-3-tyl. The heterocycle groups of the present invention are optionally substituted with one, two, three, four, or five substituents.

“Heterocycle” includes by way of example and not limitation these heterocycles described in Paquette, Leo A.; Principles of Modern Heterocyclic Chemistry (W. A. Benjamin, N.Y., 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. In one specific embodiment of the invention “heterocycle” includes a “carbocycle” as defined herein, wherein one or more (e.g. 1, 2, 3, or 4) carbon atoms have been replaced with a heteroatom (e.g. O, N, or S).

Examples of heterocycles include by way of example and not limitation pyridyl, dihydropyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4H-carbazolyl, carbazolyl, f3-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl:

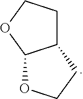

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or .beta.-carboline. Still more typically, nitrogen bonded heterocycles include 1-aziridyl, 1-azetedyl, 1-pyrrolyl, 1-imidazolyl, 1-pyrazolyl, and 1-piperidinyl.

As used herein, “cycloalkyl” refers to refers to a saturated monocyclic, hydrocarbon ring system having three to twelve carbon atoms and zero heteroatoms. Representative examples of cycloalkyl groups include, but are not limited to, cyclopropyl, cyclopentyl, and cyclohexyl. The cycloalkyl groups of the present invention are optionally substituted with one, two, three, four, or five substituents.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-fungal or anti-bacterial activity using the standard tests described herein, or using other similar tests which are well known in the art.

“Therapeutically effective amount” is intended to include an amount of a compound described herein, or an amount of the combination of compounds described herein, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes: (i) preventing a pathologic condition from occurring (e.g. prophylaxis); (ii) inhibiting the pathologic condition or arresting its development; (iii) relieving the pathologic condition; and/or (iv) diminishing symptoms associated with the pathologic condition.

The compounds described herein, post administration, can hydrolyze to provide the active ingredient to the intended target. “Hydrolysis in Drug and Prodrug Metabolism: Chemistry, Biochemistry, and Enzymology,” by Bernard Testa and Joachim Mayer; Wiley-VCH (August 2003) provides a comprehensive review of metabolic reactions and enzymes involved in the hydrolysis of pharmaceutical compounds. The text also describes the significance of biotransformation and discusses the physiological roles of hydrolytic enzymes, hydrolysis of amides, and the hydrolysis of lactams. Additional references useful in designing compounds disclosed herein include, e.g., “Biological Approaches to the Controlled Delivery of Drugs,” Annals of the New York Academy of Sciences, Vol. 507, R. L. Juliano ed., (December 1987); Amer. Biological Agent Assn., “Design of Biopharmaceutical Properties through Prodrugs and Analogs” Edward B. Roche ed., (June 1977); Marcel Dekker, “Prodrugs: Topical and Ocular Drug Delivery,” Drugs and the Biological Agent Sciences, Vol. 53, Kenneth B. Sloan ed., (Mar. 17, 1992); “Enzyme-Prodrug Strategies for Cancer Therapy,” Roger G. Melton, Richard J. Knox eds., Kluwer Academic/Plenum Publishers (February 1999); “Design of Prodrugs,” Hans Bundgaard ed., Elsevier Science (November 1985); “Textbook of Drug Design and Development,” Povl Krogsgaard-Larsen, Hans Bundgaard eds., Hardwood Academic Pub (July 1991); “Conversion of Non-Toxic Prodrugs to Active, Anti-Neoplastic Drugs Selectively in Breast Cancer Metastases,” Basse, Per H. (September 2000); and Masson et al., “Marine lipids for prodrugs, soft compounds and other pharmaceutical applications,” Die Pharmazie, 55(3):172-7 (March 2000).

The term “tumor” is commonly used as a synonym for a neoplasm (a solid or fluid-filled [cystic] lesion that may or may not be formed by an abnormal growth of neoplastic cells) that appears enlarged in size. Tumor is not synonymous with cancer. While cancer is by definition malignant, a tumor can be benign, pre-malignant, or malignant, or can represent a lesion without any cancerous potential whatsoever.

The term “benign tumor” refers to a mass of cells (tumor) that lacks the ability to invade neighboring tissue or metastasize. These characteristics are required for a tumor to be defined as cancerous and therefore benign tumors are non-cancerous. Also, benign tumors generally have a slower growth rate than malignant tumors and the tumor cells are usually more differentiated (cells have normal features).Benign tumors are typically surrounded by an outer surface (fibrous sheath of connective tissue) or remain with the epithelium. Common examples of benign tumors include moles (nevi) and uterine fibroids (leiomyomas).

The term “malignant tumor” refers to a mass of cells (tumor) that possesses the ability to invade neighboring tissue or metastasize.

The term “cancer” refers to a broad group of diseases involving unregulated cell growth. In cancer, cells divide and grow uncontrollably, forming malignant tumors, and invade nearby parts of the body. The cancer may also spread to more distant parts of the body through the lymphatic system or bloodstream. Not all tumors are cancerous; benign tumors do not invade neighboring tissues and do not spread throughout the body. There are over 200 different known cancers that afflict humans.

The term “liver cancer” or “hepatic cancer” refers to a cancer that originates in the liver. Liver cancers are malignant tumors that grow on the surface or inside the liver. Liver tumors are discovered on medical imaging equipment (often by accident) or present themselves symptomatically as an abdominal mass, abdominal pain, jaundice, nausea or liver dysfunction. Liver cancers should not be confused with liver metastases, which are cancers that originate from organs elsewhere in the body and migrate to the liver.

The term “hepatocellular carcinoma (HCC)” refers to the most common type of liver cancer. Most cases of HCC are secondary to either a viral hepatitis infection (hepatitis B or C) or cirrhosis (alcoholism being the most common cause of hepatic cirrhosis).

The term “palliative management” refers to (i) providing a treatment designed to prolong the life of the patient, (ii) providing treatment designed to relieve pain and distress with no attempt to cure, or (iii) focusing on improving a patient's quality of life-not just in the body, but also in your mind and spirit. Sometimes palliative care is combined with curative treatment.

The term “adenocarcinoma” refers to a cancer of an epithelium that originates in glandular tissue. Epithelial tissue includes, but is not limited to, the surface layer of skin, glands, and a variety of other tissue that lines the cavities and organs of the body. Epithelium can be derived embryologically from ectoderm, endoderm or mesoderm. To be classified as Adenocarcinoma, the cells do not necessarily need to be part of a gland, as long as they have secretory properties. Well differentiated adenocarcinomas tend to resemble the glandular tissue that they are derived from, while poorly differentiated adenocarcinomas may not.

The term “gastrointestinal” refers to all the anatomical structures from the mouth to the anus.

The term “metastatic” refers to the spread of a cancer from one organ or part to another non-adjacent organ or part. The new occurrences of disease thus generated are referred to as metastases (sometimes abbreviated mets).

The term “hepatitis C virus (HCV)” refers to a small (55-65 nm in size), enveloped, positive-sense single-stranded RNA virus of the family Flaviviridae. Hepatitis C virus is the cause of hepatitis C in humans.

The term “in vitro” refers to activities that are conducted using components of an organism that have been isolated from their usual biological surroundings in order to permit a more detailed or more convenient analysis than can be done with whole organisms.

The term “in vivo” refers to activities using a whole, living organism as opposed to a partial or dead organism.

The term “antimetabolite” refers to chemical that inhibits the use of a metabolite, which is another chemical that is part of normal metabolism. Such substances are often similar in structure to the metabolite that they interfere with, such as the antifolates that interfere with the use of folic acid. The presence of antimetabolites can have toxic effects on cells, such as halting cell growth and cell division, so these compounds are used as chemotherapy for cancer.

The term “Capecitabine” refers to the compound chemically designated as pentyl [1-(3,4-dihydroxy-5-methyltetrahydrofuran-2-yl)-5-fluoro-2-oxo-1H-pyrimidin-4-yl]carbamate, structurally shown below:

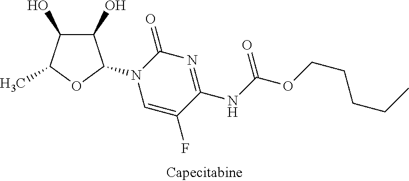 [SMILES: CCCCCOC(=O)Nc1nc(=O)n([C@@H]2O[C@H](C)[C@@H](O)[C@H]2O)cc1F]

The term “Gemcitabine” refers to the compound chemically designated as 4-amino-1-(2-deoxy-2,2-difluoro-β-D-erythro-pentofuranosyl)pyrimidin-2(1H)-on, structurally shown below:

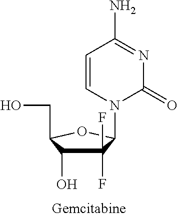

The term “Cytarabine” refers to the compound chemically designated as 4-amino-1-[(2R,3S,4R,5R)-3,4-dihydroxy-5-(hydroxymethyl)oxolan-2-yl]pyrimidin-2-one, structurally shown below:

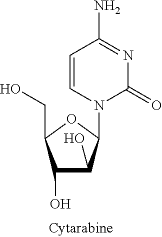

The term “Pentostatin” refers to the compound chemically designated as (R)-3-((2R,4S,5R)-4-hydroxy-5-(hydroxymethyl)tetrahydrofuran-2-yl)-3,6,7,8-tetrahydroimidazo[4,5-d][1,3]diazepin-8-ol, structurally shown below:

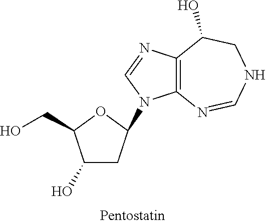 [SMILES: OC[C@H]1O[C@@H](n2cnc3c2N=CNC[C@H]3O)C[C@@H]1O]

The term “Fludarabine” refers to the compound chemically designated as [(2R,3R,4S,5R)-5-(6-amino-2-fluoro-purin-9-yl)-3,4-dihydroxy-oxolan-2-yl]hydroxyl methyl, structurally shown below:

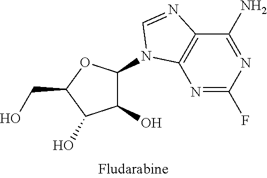 [SMILES: Nc1nc(F)nc2c1ncn2[C@@H]1O[C@H](CO)[C@@H](O)[C@@H]1O]

The term “Cladribine” refers to the compound chemically designated as 5-(6-amino-2-chloro-purin-9-yl)-2-(hydroxymethyl)oxolan-3-ol, structurally shown below:

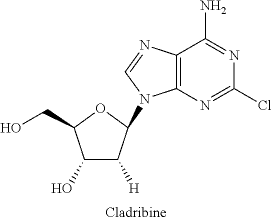

The term “Clofarabine” refers to the compound chemically designated as 5-(6-amino-2-chloro-purin-9-yl) -4-fluoro-2-(hydroxymethyl)oxolan-3-ol, structurally shown below:

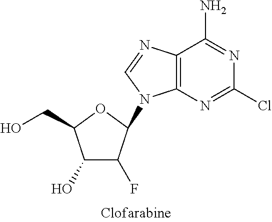 [SMILES: Nc1nc(Cl)nc2c1ncn2[C@@H]1O[C@H](CO)[C@@H](O)C1F]

The term “Methotrexate” refers to the compound chemically designated as (2S)-2-[(4-{[(2,4-diaminopteridin-6-yl)methyl](methyl)amino}benzoyl)amino]pentanedioic acid, structurally shown below:

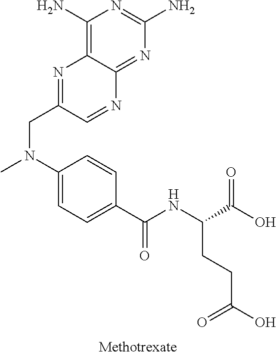 [SMILES: CN(Cc1cnc2nc(N)nc(N)c2n1)c1ccc(C(=O)N[C@@H](CCC(=O)O)C(=O)O)cc1]

The term “Decitabine” refers to the compound chemically designated as 4-Amino-1-(2-deoxy-β-D-erythro-pentofuranosyl)-1,3,5-triazin-2(1H)-one, structurally shown below:

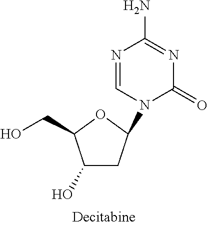 [SMILES: Nc1ncn([C@H]2C[C@H](O)[C@@H](CO)O2)c(=O)n1]

The term “Epirubicin” refers to the compound chemically designated as (8R,10S)-10-((2S,4S,5R,6S)-4-amino-5-hydroxy-6-methyltetrahydro-2H-pyran-2-yl)-6,8,11-trihydroxy-8-(2-hydroxyacetyl)-1-methoxy-7,8,9,10-tetrahydrotetracene-5,12-dione, structurally shown below:

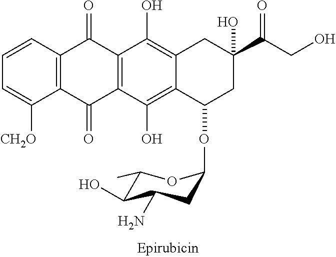

The term “Raloxifene” refers to the compound chemically designated as [6-hydroxy-2-(4-hydroxyphenyl)-benzothiophen-3-yl]-[4-[2-(1-piperidyl)ethoxy]phenyl]-methanone, structurally shown below:

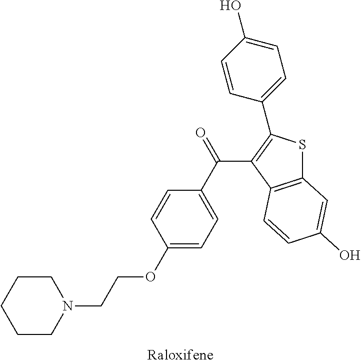 [SMILES: O=C(c1ccc(OCCN2CCCCC2)cc1)c1c(-c2ccc(O)cc2)sc2cc(O)ccc12]

The term “Azacitidine” refers to the compound chemically designated as 4-amino-1-β-D-ribofuranosyl-1,3,5-triazin-2(1H)-one, structurally shown below:

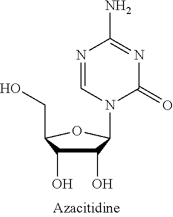

The term “Ribavirin” refers to the compound chemically designated as 1-[(2R,3R,4S,5R)-3,4-dihydroxy-5-(hydroxymethyl)oxolan-2-yl]-1H-1,2,4-triazole-3-carboxamide, structurally shown below:

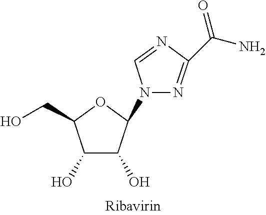 [SMILES: NC(=O)c1ncn([C@@H]2O[C@H](CO)[C@@H](O)[C@H]2O)n1]

The term “monovalent” refers to an atom, ion, or chemical group with a valence of one, which thus can form one covalent bond.

The term “substituent group” (sometimes referred to as a “radical”) refers to an atom, molecule, or ion that has unpaired valence electrons or an open electron shell, and therefore may be seen as having one or more “dangling” covalent bonds. A notable example of substituent group is the hydroxyl substituent group (HO—).

### Methods of Manufacturing

The compounds disclosed can be prepared by any of the applicable techniques of organic synthesis. Many such techniques are well known in the art. However, many of the known techniques are elaborated in Compendium of Organic Synthetic Methods (John Wiley & Sons, New York) Vol. 1, Ian T. Harrison and Shuyen Harrison (1971); Vol. 2, Ian T. Harrison and Shuyen Harrison (1974); Vol. 3, Louis S. Hegedus and Leroy Wade (1977); Vol. 4, Leroy G. Wade Jr., (1980); Vol. 5, Leroy G. Wade Jr. (1984); and Vol. 6, Michael B. Smith; as well as March, J., Advanced Organic Chemistry, 3rd Edition, John Wiley & Sons, New York (1985); Comprehensive Organic Synthesis. Selectivity, Strategy & Efficiency in Modern Organic Chemistry, In 9 Volumes, Barry M. Trost, Editor-in-Chief, Pergamon Press, New York (1993); Advanced Organic Chemistry, Part B: Reactions and Synthesis, 4th Ed.; Carey and Sundberg; Kluwer Academic/Plenum Publishers: New York (2001); Advanced Organic Chemistry, Reactions, Mechanisms , and Structure, 2nd Edition, March, McGraw Hill (1977); Protecting Groups in Organic Synthesis, 2nd Edition, Greene, T.W., and Wutz, P.G.M., John Wiley & Sons, New York (1991); and Comprehensive Organic Transformations, 2nd Edition, Larock, R.C., John Wiley & Sons, New York (1999).

Further description for the methods of preparing the compounds described herein can be found in the examples herein.

The compounds described herein can be purified utilizing standard, well-known purification techniques of organic synthesis. Such purification techniques include, e.g., sublimation, crystallization, filtration, extraction (e.g., liquid-liquid extraction or solid-liquid extraction), distillation (e.g., fractional distillation), and chromatography (e.g., high pressure liquid chromatography (HPLC)).

### Pharmaceutical Formulations

The compounds described herein can be formulated with conventional carriers and excipients, which will be selected in accord with ordinary practice. Tablets can contain excipients, glidants, fillers, binders and the like. Aqueous formulations can be prepared in sterile form, and when intended for delivery by other than oral administration generally will be isotonic. All formulations can optionally contain excipients such as those set forth in the Handbook of Pharmaceutical Excipients, 5^{th} Ed.; Rowe, Sheskey, and Owen, Eds.; American Pharmacists Association; Pharmaceutical Press: Washington, D.C., 2006. Excipients can include ascorbic acid and other antioxidants, chelating agents such as EDTA, carbohydrates such as dextrin, hydroxyalkylcellulose, hydroxyalkylmethylcellulose, stearic acid and the like. The pH of the formulations can range from about 3 to about 11, but will ordinarily be about 7 to 10.

While it is possible for the compounds described herein to be administered alone it may be preferable to present them as pharmaceutical formulations. The formulations, both for veterinary and for human use, include at least one compound described herein, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences, Mack Publishing Company, Easton, Pa., (1985). Such methods include the step of bringing into association the compound described herein with the carrier, which constitutes one or more accessory ingredients. In general the formulations are prepared by uniformly and intimately bringing into association the compound described herein, with liquid carriers or finely divided solid carriers or both, and then, if necessary, shaping the product.

Formulations of the present invention suitable for oral administration may be presented as discrete units such as capsules, cachets or tablets each containing a predetermined amount of the compound described herein; as a powder or granules; as a solution or a suspension in an aqueous or non-aqueous liquid; or as an oil-in-water liquid emulsion or a water-in-oil liquid emulsion. The compound described herein may also be administered as a bolus, electuary or paste.

A tablet is made by compression or molding, optionally with one or more accessory ingredients. Compressed tablets may be prepared by compressing in a suitable machine the compound described herein in a free-flowing form such as a powder or granules, optionally mixed with a binder, lubricant, inert diluent, preservative, surface active or dispersing agent. Molded tablets may be made by molding in a suitable machine a mixture of the powdered compound described herein moistened with an inert liquid diluent. The tablets may optionally be coated or scored and optionally are formulated so as to provide slow or controlled release of the compound described herein therefrom.

For administration to the eye or other external tissues e.g., mouth and skin, the formulations are preferably applied as a topical ointment or cream containing the compound(s) described herein in an amount of, for example, 0.075 to 20% w/w (including compound(s) described herein in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the compound(s) described herein may be employed with either a paraffinic or a water-miscible ointment base. Alternatively, the compound(s) described herein may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the compound(s) described herein through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention include one or more compounds described herein with one or more pharmaceutically acceptable carriers or excipients and optionally other therapeutic agents. Pharmaceutical formulations containing the compound(s) described herein may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the compound(s) described herein in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the compound described herein is mixed with an inert solid diluent, for example calcium phosphate or kaolin, or as soft gelatin capsules wherein the compound described herein is mixed with water or an oil medium, such as peanut oil, liquid paraffin or olive oil.

Aqueous suspensions of the invention contain the compound(s) described herein in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the compound(s) described herein in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the compound(s) described herein in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of compound(s) described herein that may be combined with the carrier material to produce a single dosage form will vary depending upon the host treated and the particular mode of administration. For example, a time-release formulation intended for oral administration to humans may contain approximately 1 to 1500 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the compound(s) described herein per milliliter of solution in order that infusion of a suitable volume at a rate of about 30 mL/hr can occur.

Formulations suitable for administration to the eye include eye drops wherein the compound described herein is dissolved or suspended in a suitable carrier, especially an aqueous solvent for the compound(s) described herein. The compound described herein is preferably present in such formulations in a concentration of 0.5 to 20%, advantageously 0.5 to 10% particularly about 1.5% w/w.

Formulations suitable for topical administration in the mouth include lozenges comprising the compound(s) described herein in a flavored basis, usually sucrose and acacia or tragacanth; pastilles comprising the compound(s) described herein in an inert basis such as gelatin and glycerin, or sucrose and acacia; and mouthwashes comprising the compound(s) described herein in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the compound(s) described herein. Formulations suitable for aerosol or dry powder administration may be prepared according to conventional methods and may be delivered with other therapeutic agents such as compounds heretofore used in the treatment or prophylaxis of a given condition.

Formulations suitable for vaginal administration may be presented as pessaries, tampons, creams, gels, pastes, foams or spray formulations containing in addition to the compound(s) described herein such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the compound(s) described herein.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one compound described herein as above defined together with a veterinary carrier therefor.

Veterinary carriers are materials useful for the purpose of administering the composition and may be solid, liquid or gaseous materials which are otherwise inert or acceptable in the veterinary art and are compatible with the compound(s) described herein. These veterinary compositions may be administered orally, parenterally or by any other desired route.

Compounds of the invention can also be formulated to provide controlled release of the compound(s) described herein to allow less frequent dosing or to improve the pharmacokinetic or toxicity profile of the compound(s) described herein. Accordingly, the invention also provided compositions comprising one or more compounds of the invention formulated for sustained or controlled release.

Effective dose of the compound(s) described herein depends at least on the nature of the condition being treated, toxicity, whether the compound is being used prophylactically (lower doses), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1500 mg, preferably between 5 mg and 1000 mg, and may take the form of single or multiple doses.

### Routes of Administration

One or more compounds of the invention (herein referred to as the compound(s) described herein) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. One advantage of the compounds of this invention is that in specific embodiments, the compounds are orally bioavailable and can be dosed orally.

### Combination Therapy

Compound(s) described herein can also be used in combination with other active ingredients. Such combinations are selected based on the condition to be treated, cross-reactivities of ingredients and pharmaco-properties of the combination.

It is also possible to combine any compound described herein with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide “synergy” and “synergistic effect”, i.e. the effect achieved when the compounds used together is greater than the sum of the effects that results from using the compounds separately. A synergistic effect may be attained when the compounds are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each compound described herein is administered sequentially, i.e. serially, whereas in combination therapy, effective dosages of two or more compounds described herein are administered together.

Pharmaceutical kits useful in the present invention, which include a therapeutically effective amount of a pharmaceutical composition that includes a compound of component (a) and one or more compounds of component (b), in one or more sterile containers, are also within the ambit of the present invention. Sterilization of the container may be carried out using conventional sterilization methodology well known to those skilled in the art. Component (a) and component (b) may be in the same sterile container or in separate sterile containers. The sterile containers or materials may include separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

### Utility

The compound(s) described herein can be useful to deliver active ingredients (e.g., chemotherapeutic agents or diagnostic agents) to the intended target. Specifically, such compound(s) can be delivered to the intended target wherein the compound hydrolyzes to provide the active ingredient (e.g., chemotherapeutic agent) to the intended target. This is accomplished, e.g., by employing a specific phosphoramidate or phosphoramidite group, which is covalently conjugated to a chemotherapeutic agent or antineoplastic agent.

All publications, patents, and published patent applications disclosed herein are incorporated herein by reference in their entirety. While in the foregoing specification this invention (as defined by the issued claims) invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

### Specific Values

Specific values listed below for substituent groups, substituents, and ranges, are for illustration only. They do not exclude other defined values or other values within defined ranges for the substituent groups and substituents.

With the compound of formula (I), a specific value for R¹ is a nitrogenous base selected from the group consisting of adenine, guanine, thymidine, cytosine, and uracil, optionally substituted by 1, 2, or 3 U; wherein each U is independently halo, hydroxy, alkoxy, trifluoromethyl, or hydroxyl alkyl.

With the compound of formula (I), a specific value for R¹ is uracil optionally substituted by 1, 2, or 3 U; wherein each U is independently halo, hydroxy, alkoxy, trifluoromethyl, or hydroxyl alkyl

With the compound of formula (I), a specific value for R¹ is uracil substituted by 1 U; wherein U is halo.

With the compound of formula (I), a specific value for R¹ is uracil substituted by 1 U; wherein U is fluoro.

With the compound of formula (I), a specific value for R¹ is a compound of formula (Ib):

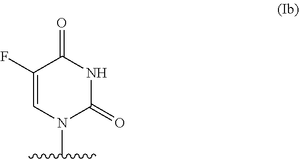 [SMILES: *n1cc(F)c(=O)[nH]c1=O]

With the compound of formula (I), a specific value for R⁴ is halo, hydroxy, alkoxy, trifluoromethyl, or hydroxyl alkyl.

With the compound of formula (I), a specific value for R⁴ is hydroxyl.

With the compound of formula (I), a specific value for R² is hydrogen.

With the compound of formula (I), a specific value for R³ is hydrogen.

With the compound of formula (I), a specific value for R⁵ is hydrogen.

With the compound of formula (I), a specific value for R⁶ is hydrogen.

With the compound of formula (I), a specific value for R⁷ is H, alkyl, cycloalkyl or aryl.

With the compound of formula (I), a specific value for R⁷ is H.

With the compound of formula (I), a specific value for R⁷ is aryl.

With the compound of formula (I), a specific value for R⁷ is phenyl.

With the compound of formula (I), a specific value for R⁷ is cycloalkyl.

With the compound of formula (I), a specific value for R⁸ is hydrogen or alkyl.

With the compound of formula (I), a specific value for R⁸ is hydrogen.

With the compound of formula (I), a specific value for R⁹-R¹¹ is that each is independently hydrogen or alkyl.

With the compound of formula (I), a specific value for R⁹ is alkyl.

With the compound of formula (I), a specific value for R⁹ is isopropyl.

With the compound of formula (I), a specific value for R¹⁰ and R¹¹ is that one of R¹⁰ and R¹¹ is hydrogen and the other is alkyl.

With the compound of formula (I), a specific value for R¹⁰ and R¹¹ is that one of R¹⁰ and R¹¹ is hydrogen and the other is methyl.

With the compound of formula (I), a specific value for X^{O} is O or absent.

With the compound of formula (I), a specific value for X^{O} is O.

With the compound of formula (I), a specific value for X^{O} is absent (e.g., the P atom has a valence of (III)).

With the compound of formula (I), a specific value for X is O or NR^{X}, wherein RX is hydrogen or alkyl.

With the compound of formula (I), a specific value for X is O.

With the compound of formula (I), a specific value for X is NR^{X}, wherein R^{X} is hydrogen or alkyl.

With the compound of formula (I), a specific value for X is NR^{X}, wherein R^{X} is hydrogen.

With the compound of formula (I), a specific value for X is NR^{X}, wherein R^{X} is alkyl.

With the compound of formula (II), a specific value for R¹²-R¹⁶, R¹⁸-R²¹, R^{a} and R^{b} is that each is independently hydrogen or alkyl.

With the compound of formula (II), a specific value for R¹² is alkyl.

With the compound of formula (II), a specific value for R¹² is methyl.

With the compound of formula (II), a specific value for R¹³ is hydrogen.

With the compound of formula (II), a specific value for R¹⁴ is hydrogen.

With the compound of formula (II), a specific value for R¹⁵ is hydrogen.

With the compound of formula (II), a specific value for R^{a} is hydrogen.

With the compound of formula (II), a specific value for R^{b} is hydrogen.

With the compound of formula (II), a specific value for R¹⁶ is alkyl.

With the compound of formula (II), a specific value for R¹⁶ is methyl.

With the compound of formula (II), a specific value for R¹⁷ is aryl, cycloalkyl, heterocycle, heteroaryl, aryl alkyl, cycloalkyl alkyl, heterocycle alkyl, or heteroaryl alkyl, optionally substituted by 1, 2, or 3 Y; wherein each Y is independently halo, hydroxy, nitro, cyano, alkoxy, trifluoromethyl, or hydroxyl alkyl.

With the compound of formula (II), a specific value for R¹⁷ is aryl.

With the compound of formula (II), a specific value for R¹⁷ is phenyl.

With the compound of formula (II), a specific value for R¹⁸ is hydrogen.

With the compound of formula (II), a specific value for R¹⁹ is alkyl.

With the compound of formula (II), a specific value for R¹⁹ is isopropyl.

With the compound of formula (II), a specific value for R²⁰ and R²¹ is that one of R²⁰ and R²¹ is hydrogen and the other is alkyl.

With the compound of formula (II), a specific value for R²⁰ and R²¹ is that one of R²⁰ and R²¹ is hydrogen and the other is methyl.

With the compound of formula (II), a specific value for X^{O} is O or absent.

With the compound of formula (II), a specific value for X^{O} is O.

With the compound of formula (II), a specific value for X^{O} is absent (e.g., the phosphorus atom has a valence of (III)).

One preferred embodiment is a compound of formula (III):

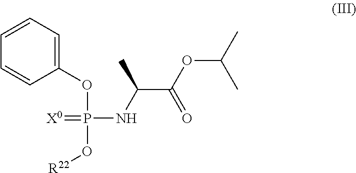

wherein,

R²² is a monovalent substituent group of a chemotherapeutic agent, or is a monovalent substituent group of a diagnostic agent; and

X^{O} is O or absent.

In a preferred embodiment, X^{O} is O.

One preferred embodiment is a method for targeting a tumor or cancer cell, the method including contacting mammalian tissue with a compound of formula (III):

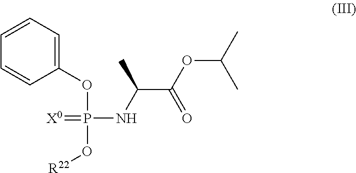

or a pharmaceutically acceptable thereof, wherein,

R²² is a monovalent substituent group of a chemotherapeutic agent, or is a monovalent substituent of an antiviral agent, or is a monovalent substituent group of a diagnostic agent; and

X^{O} is O or absent.

In a preferred embodiment, X^{O} is O.

Sofosbuvir is a compound of formula (III) that is an effective drug for treating hepatitis C.

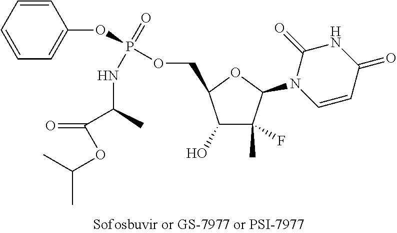 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

PSI-7851 is a racemic mixture, while sofosbuvir is the same compound as a pure optical isomer.

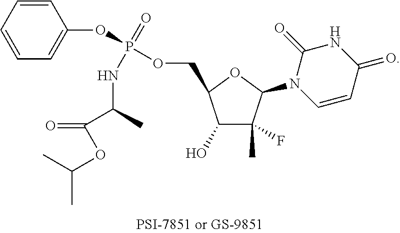 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

In a single ascending dose clinical study of healthy volunteers receiving doses up to 800 mg once daily, PSI-7851 was demonstrated to be generally safe and well tolerated, with no dose-limiting toxicities. In addition, pharmacokinetic results exhibited a systemic exposure profile that was consistent with rapid uptake of the drug by the liver and low plasma exposure to the prodrug PSI-7851 (Denning, J. et al., 2013, Pharmacokinetics, Safety, and Tolerability of GS-9851, a Nucleotide Analog Polymerase Inhibitor for Hepatitis C Virus, following Single Ascending Doses in Healthy Subjects, Antimicrobial Agents and Chemotherapy 57:1201-1208).

Although it has not previously been suggested, the inventor believes this specific uptake by the liver is not limited to PSI-7851 but is a property of all compounds of formula (III), especially where X^{O} is O. Thus, the compounds of formula III where R²² is a chemotherapeutic agent or antiviral agent are effective compounds for treating liver disease, particularly liver cancer and hepatitis.

Thus, the inventor believes that compounds of formula (III), and particularly the compound of formula (Ic) will have excellent uptake and targeting to the liver and to hepatocellular carcinoma cells. In particular, the compound of formula (Ic) will have excellent uptake by the liver and targeting to the liver for effective treatment of hepatocellular carcinoma. The compound of formula (Ic) is a compound of formula (III) where R²² is fluorodeoxyuridine (FUDR) and X^{O} is O.

Thus, although the compound of formula (Ic) can be given orally, it accumulates in liver due to the first-pass effect. Therefore it provides very high concentrations of Floxuridine in the liver. Furthermore this targeting also increases uptake of the drug by tumor cells. Additionally FUDR itself is almost totally extracted by liver. Thus there is a triple targeting mechanism inbuilt in this new chemical entity.

The demonstration that PSI-7851 can be given orally at doses of 800 mg/day to healthy volunteers suggests that the compound of formula (Ic) can be given orally at higher doses than FUDR is given. FUDR is given at 0.18 mg/kg/day.

In specific embodiments of the method of treating hepatocellular carcinoma comprising administering a compound of formula (Ic) or pharmaceutical salt thereof, the compound of formula (Ic) is administered at a dosage of 0.2 mg/kg/day or more, 1 mg/kg/day or more, 2 mg/kg/day or more, or 5 mg/kg/day or more. In specific embodiments, it is administered at a dosage of 0.2 to 4, 0.2 to 10, 0.2 to 20, 0.2 to 100, 1 to 4, 1 to 20, 1 to 100, 2 to 4, 2 to 10, 2 to 20, 2 to 100, 5 to 10, 5 to 20, or 5 to 100 mg/kg/day.

With the compound of formula (III), specific values for the substituents on phosphoramidate or phosphoramidite group can be any one or more from R⁷, R⁸, R⁹, R¹⁰ and R¹¹.

With the compound of formula (III), a specific value for X^{O} is O or absent.

With the compound of formula (III), a specific value for X^{O} is O.

With the compound of formula (III), a specific value for X^{O} is absent (e.g., the phosphorus atom has a valence of (III).

With the compound of formula (III), a specific value for R²² is a monovalent substituent group of a chemotherapeutic agent, or is a monovalent substituent group of a diagnostic agent.

With the compound of formula (III), a specific value for R²² is a monovalent substituent group of a chemotherapeutic agent.

With the compound of formula (III), a specific value for R²² is a monovalent substituent group of a diagnostic agent.

With the compound of formula (III), a specific value for the chemotherapeutic agent is Capecitabine.

With the compound of formula (III), a specific value for the chemotherapeutic agent is Gemcitabine.

With the compound of formula (III), a specific value for the chemotherapeutic agent is Cytarabine.

With the compound of formula (III), a specific value for the chemotherapeutic agent is Pentostatin.

With the compound of formula (III), a specific value for the chemotherapeutic agent is Fludarabine.

With the compound of formula (III), a specific value for the chemotherapeutic agent is Cladribine.

With the compound of formula (III), a specific value for the chemotherapeutic agent is Clofarabine.

With the compound of formula (III), a specific value for the chemotherapeutic agent is Methotrexate.

With the compound of formula (III), a specific value for the chemotherapeutic agent is Decitabine.

With the compound of formula (III), a specific value for the chemotherapeutic agent is Epirubicin.

With the compound of formula (III), a specific value for the chemotherapeutic agent is Raloxifene.

With the compound of formula (III), a specific value for the chemotherapeutic agent is Azacitidine.

With the compound of formula (III), a specific value for the chemotherapeutic agent is Ribavirin.

With the compound of formula (III), a specific value for the monovalent substituent group of the chemotherapeutic agent is

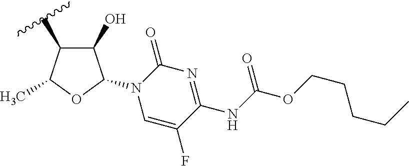 [SMILES: *[C@H]1[C@@H](O)[C@H](n2cc(F)c(NC(=O)OCCCCC)nc2=O)O[C@@H]1C]

With the compound of formula (III), a specific value for the monovalent substituent group of the chemotherapeutic agent is

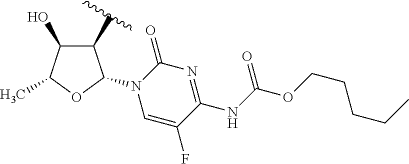 [SMILES: *[C@@H]1[C@H](O)[C@@H](C)O[C@H]1n1cc(F)c(NC(=O)OCCCCC)nc1=O]

With the compound of formula (III), a specific value for the monovalent substituent group of the chemotherapeutic agent is

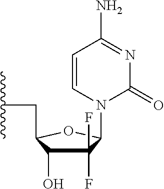

With the compound of formula (III), a specific value for the monovalent substituent group of the chemotherapeutic agent is

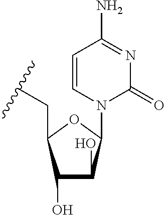

With the compound of formula (III), a specific value for the monovalent substituent group of the chemotherapeutic agent is

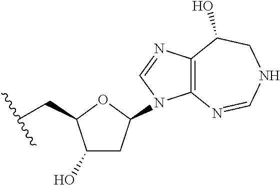 [SMILES: *C[C@H]1O[C@@H](n2cnc3c2N=CNC[C@H]3O)C[C@@H]1O]

With the compound of formula (III), a specific value for the monovalent substituent group of the chemotherapeutic agent is

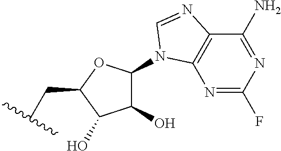 [SMILES: *C[C@H]1O[C@@H](n2cnc3c(N)nc(F)nc32)[C@@H](O)[C@@H]1O]

With the compound of formula (III), a specific value for the monovalent substituent group of the chemotherapeutic agent is

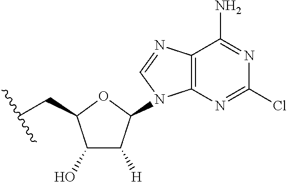

With the compound of formula (III), a specific value for the monovalent substituent group of the chemotherapeutic agent is

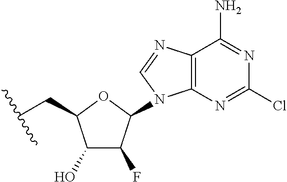 [SMILES: *C[C@H]1O[C@@H](n2cnc3c(N)nc(Cl)nc32)[C@@H](F)[C@@H]1O]

With the compound of formula (III), a specific value for the monovalent substituent group of the chemotherapeutic agent is

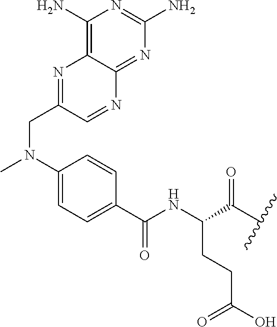 [SMILES: *C(=O)[C@H](CCC(=O)O)NC(=O)c1ccc(N(C)Cc2cnc3nc(N)nc(N)c3n2)cc1]

With the compound of formula (III), a specific value for the monovalent substituent group of the chemotherapeutic agent is

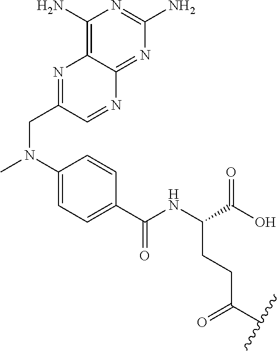 [SMILES: *C(=O)CC[C@H](NC(=O)c1ccc(N(C)Cc2cnc3nc(N)nc(N)c3n2)cc1)C(=O)O]

With the compound of formula (III), a specific value for the monovalent substituent group of the chemotherapeutic agent is

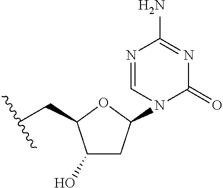 [SMILES: *C[C@H]1O[C@@H](n2cnc(N)nc2=O)C[C@@H]1O]

With the compound of formula (III), a specific value for the monovalent substituent group of the chemotherapeutic agent is

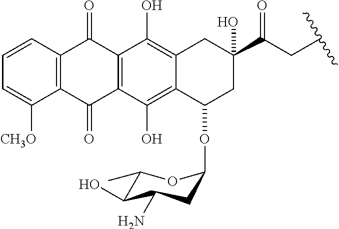

With the compound of formula (III), a specific value for the monovalent substituent group of the chemotherapeutic agent is

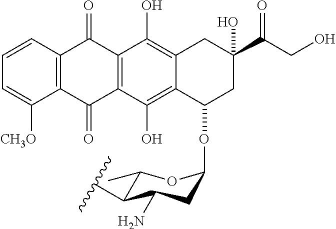

With the compound of formula (III), a specific value for the monovalent substituent group of the chemotherapeutic agent is

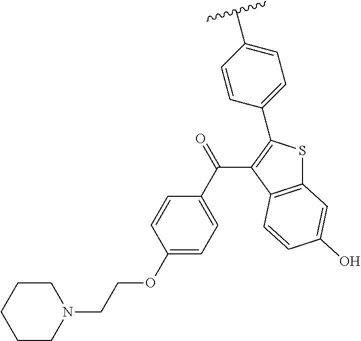 [SMILES: *c1ccc(-c2sc3cc(O)ccc3c2C(=O)c2ccc(OCCN3CCCCC3)cc2)cc1]

With the compound of formula (III), a specific value for the monovalent substituent group of the chemotherapeutic agent is

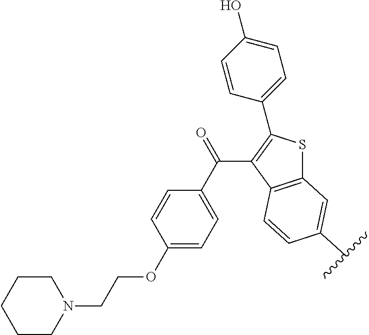 [SMILES: *c1ccc2c(C(=O)c3ccc(OCCN4CCCCC4)cc3)c(-c3ccc(O)cc3)sc2c1]

With the compound of formula (III), a specific value for the monovalent substituent group of the chemotherapeutic agent is

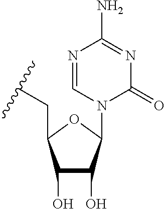

With the compound of formula (III), a specific value for the monovalent substituent group of the chemotherapeutic agent is

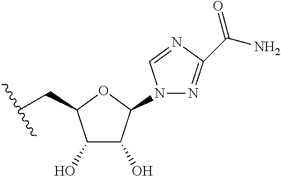 [SMILES: *C[C@H]1O[C@@H](n2cnc(C(N)=O)n2)[C@H](O)[C@@H]1O]

In specific embodiments, the administration is oral.

In specific embodiments, the dosage is about 0.05 mg/kg/day to about 20 mg/kg/day.

In specific embodiments, the cancer includes a solid tumor.

In specific embodiments, the treating the cancer includes at least one of reducing the size of a solid tumor and slowing the growth of a solid tumor.

In specific embodiments, the cancer includes a benign or malignant tumor.

In specific embodiments, the cancer includes a benign tumor.

In specific embodiments, the cancer includes a malignant tumor.

In specific embodiments, the cancer includes individual cancel cells or colonies of cancer cells.

In specific embodiments, the cancer is located in at least one of the lung, female breast, colon, stomach, prostate, liver, cervix, esophagus, bladder, uterus, pancreas, kidney, brain, ovary, thyroid, red blood cells, lymphatic system, mouth, lip, brain, skin and larynx.

In specific embodiments, the cancer is hepatocellular carcinoma (HCC).

In specific embodiments, the cancer is liver cancer.

In specific embodiments, the patient is considered incurable by surgery.

In specific embodiments, the method is carried out with the co-administration of one or more additional active pharmaceutical ingredients (APIs).

In specific embodiments, the method is carried out with the co-administration of one or more additional chemotherapeutic agents.

In specific embodiments, the virus is hepatitis C virus (HCV).

In specific embodiments, the compound or formulation is administered for a period of time of at least about 1 week.

In specific embodiments, the compound or formulation is administered for a period of time of at least about 1 month.

In specific embodiments, the compound or formulation is administered for a period of time of at least about 3 months.

### Enumerated Embodiments

Specific enumerated embodiments [1] to [73] provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.
[1.] A compound of formula (I):

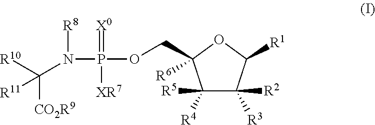

wherein:

R¹ is a nitrogenous base selected from the group consisting of adenine, guanine, thymidine, cytosine, and uracil, optionally substituted by 1, 2, or 3 U; wherein each U is independently halo, hydroxy, alkoxy, trifluoromethyl, or hydroxyl alkyl;

R², R³, R⁵, and R⁶ are each independently hydrogen;

R⁴ is halo, hydroxy, alkoxy, trifluoromethyl, or hydroxyl alkyl;

R⁷ is H, alkyl, cycloalkyl or aryl;

R⁸ is hydrogen or alkyl;

R⁹-R¹¹ are each independently hydrogen or alkyl;

X^{O} is O or absent and hence phosphorous atom is trivalent; and

X is O or NR^{X}, wherein RX is hydrogen or alkyl;

or a pharmaceutically acceptable salt thereof.
[2.] The compound of the above embodiment, wherein R¹ is uracil optionally substituted by 1, 2, or 3 U; wherein each U is independently halo, hydroxy, alkoxy, trifluoromethyl, or hydroxyl alkyl.
[3.] The compound of any one of the above embodiments, wherein R¹ is uracil substituted by 1 U; wherein U is halo.
[4.] The compound of any one of the above embodiments, wherein R¹ is uracil substituted by 1 U; wherein U is fluoro.
[5.] The compound of any one of the above embodiments, wherein R¹ is a compound of formula (Ib):

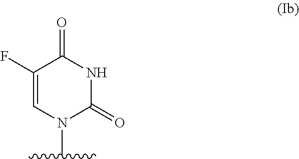 [SMILES: *n1cc(F)c(=O)[nH]c1=O]

[6.] The compound of any one of the above embodiments, wherein R⁴ is hydroxyl.
[7.] The compound of any one of the above embodiments, wherein R⁷ is aryl.
[8.] The compound of any one of the above embodiments, wherein R⁷ is phenyl.
[9.] The compound of any one of the above embodiments, wherein R⁸ is hydrogen.
[10.] The compound of any one of the above embodiments, wherein R⁹ is alkyl.
[11.] The compound of any one of the above embodiments, wherein R⁹ is isopropyl.
[12.] The compound of any one of the above embodiments, wherein one of R¹⁰ and R¹¹ is hydrogen and the other is alkyl.
[13.] The compound of any one of the above embodiments, wherein one of R¹⁰ and R¹¹ is hydrogen and the other is methyl.
[14.] The compound of any one of the above embodiments, wherein X^{O} is O.
[15.] The compound of any one of the above embodiments, wherein X^{O} is absent.
[16.] The compound of any one of the above embodiments, wherein X is O.
[17.] The compound of any one of the above embodiments, wherein X is NR^{X}, wherein R^{X} is hydrogen or alkyl.
[18.] The compound of any one of the above embodiments, wherein X is NR^{X}, wherein R^{X} is hydrogen.
[19.] The compound of any one of the above embodiments, wherein X is NR^{X}, wherein RX is alkyl.
[20.] A compound of formula (Ic):

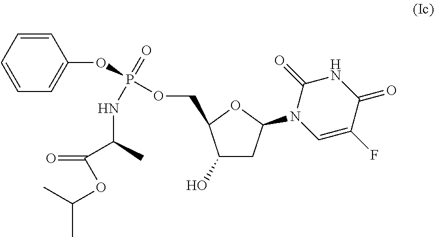 [SMILES: CC(C)OC(=O)[C@H](C)N[P@](=O)(OC[C@H]1O[C@@H](n2cc(F)c(=O)[nH]c2=O)C[C@@H]1O)Oc1ccccc1]

or a pharmaceutically acceptable salt thereof.

The compound of formula (Ic) is also known as PI-102 and as floxphosteridine.
[21.] A compound of formula (II):

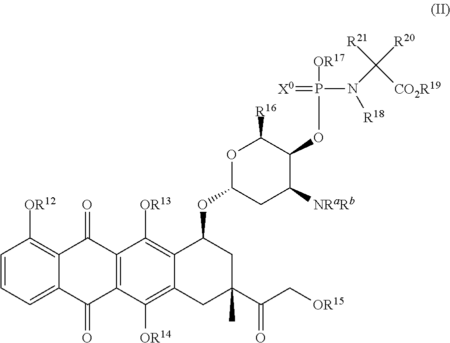

wherein,

each of R¹²-R¹⁶ , R¹⁸-R²¹, R^{a} and R^{b} is independently hydrogen or alkyl;

R¹⁷ is aryl, cycloalkyl, heterocycle, heteroaryl, aryl alkyl, cycloalkyl alkyl, heterocycle alkyl, or heteroaryl alkyl, optionally substituted by 1, 2, or 3 Y; wherein each Y is independently halo, hydroxy, nitro, cyano, alkoxy, trifluoromethyl, or hydroxyl alkyl; and

X^{O} is O or absent;

or a pharmaceutically acceptable salt thereof.
[22.] The compound of embodiment [21], wherein R¹² is alkyl.
[23.] The compound of any one of embodiments [21]-[22], wherein R¹² is methyl.
[24.] The compound of any one of embodiments [21]-[23], wherein R¹³ is hydrogen.
[25.] The compound of any one of embodiments [21]-[24], wherein R¹⁴ is hydrogen.
[26.] The compound of any one of embodiments [21]-[25], wherein R¹⁵ is hydrogen.
[27.] The compound of any one of embodiments [21]-[26], wherein R^{a} is hydrogen.
[28.] The compound of any one of embodiments [21]-[27], wherein R^{b} is hydrogen.
[29.] The compound of any one of embodiments [21]-[28], wherein R¹⁶ is alkyl.
[30.] The compound of any one of embodiments [21]-[29], wherein R¹⁶ is methyl.
[31.] The compound of any one of embodiments [21]-[30], wherein R¹⁷ is aryl.
[32.] The compound of any one of embodiments [21]-[31], wherein R¹⁷ is phenyl.
[33.] The compound of any one of embodiments [21]-[32], wherein R¹⁸ is hydrogen.
[34.] The compound of any one of embodiments [21]-[33], wherein R¹⁹ is alkyl.
[35.] The compound of any one of embodiments [21]-[34], wherein R¹⁹ is isopropyl.
[36.] The compound of any one of embodiments [21]-[35], wherein one of R²⁰ and R²¹ is hydrogen and the other is alkyl.
[37.] The compound of any one of embodiments [21]-[36], wherein one of R²⁰ and R²¹ is hydrogen and the other is methyl.
[38.] The compound of any one of embodiments [21]-[37], wherein X^{O} is O.
[39.] The compound of any one of embodiments [21]-[37], wherein X^{O} is absent.
[40.] A compound of formula (IIa):

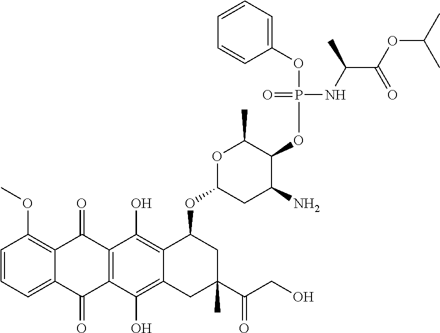 [SMILES: COc1cccc2c1C(=O)c1c(O)c3c(c(O)c1C2=O)C[C@@](C)(C(=O)CO)C[C@@H]3O[C@H]1C[C@H](N)[C@H](OP(=O)(N[C@@H](C)C(=O)OC(C)C)Oc2ccccc2)[C@H](C)O1]

or a pharmaceutically acceptable salt thereof.
[41.] A pharmaceutical composition including the compound of any one of the above embodiments, in combination with a pharmaceutically acceptable carrier.
[42.] The pharmaceutical composition of the above embodiment, suitable for oral administration.
[43.] The pharmaceutical composition of any one of the above embodiments, suitable for intravenous administration, intra muscular administration, subcutaneous administration, suppository administration, inhalation, intra nasal administration or hepatic artery infusion administration.
[44.] A method of treating cancer, the method including administering an effective amount of the compound of any one of the above embodiments, or an effective amount of the pharmaceutical composition of any one of the above embodiments, to a subject in need of such treatment, for a period of time effective to treat the cancer.
[45.] A method of treating a viral disease, the method including administering an effective amount of the compound of any one of the above embodiments, or an effective amount of the pharmaceutical composition of any one of the above embodiments, to a subject in need of such treatment, for a period of time effective to treat the viral disease.
[46.] The method of any one of the above embodiments, wherein the administration is oral.
[47.] The method of any one of the above embodiments, wherein the dosage is about 0.05 mg/kg/day to about 20 mg/kg/day.
[48.] The method of any one of the above embodiments, wherein the cancer includes a solid tumor.
[49.] The method of any one of the above embodiments, wherein the treating the cancer includes at least one of reducing the size of a solid tumor and slowing the growth of a solid tumor.
[50.] The method of any one of the above embodiments, wherein the cancer includes a benign or malignant tumor.
[51.] The method of any one of the above embodiments, wherein the cancer includes individual cancel cells or colonies of cancer cells.
[52.] The method of any one of the above embodiments, wherein the cancer is located in at least one of the lung, female breast, colon, stomach, prostate, liver, cervix, esophagus, bladder, uterus, pancreas, kidney, brain, ovary, thyroid, red blood cells, lymphatic system, mouth, lip, brain, skin and larynx.
[52b.] The method of any one of the above embodiments wherein the cancer is located in the liver.
[53.] The method of any one of the above embodiments, wherein the cancer is hepatocellular carcinoma (HCC).
[54.] A method for the palliative management of gastrointestinal adenocarcinoma metastatic to the liver, the method including administering an effective amount of the compound of any one of the above embodiments, or an effective amount of the pharmaceutical composition of any one of the above embodiments, to a subject in need of such palliative management, for a period of time effective to provide such palliative management.
[55.] The method of any one of the above embodiments, wherein such patient is considered incurable by surgery.
[56.] The method of any one of the above embodiments, carried out with the co-administration of one or more additional active pharmaceutical ingredients (APIs).
[57.] The method of any one of the above embodiments, carried out with the co-administration of one or more additional chemotherapeutic agents.
[58.] The method of any one of the above embodiments, wherein the virus is hepatitis C virus (HCV).
[59.] The method of any one of the above embodiments, wherein the period of time is at least about 1 week.
[60.] The method of any one of the above embodiments, wherein the period of time is at least about 1 month.
[61.] The method of any one of the above embodiments, wherein the period of time is at least about 3 months.
[62.] A method for targeting a tumor or cancer cell, the method including contacting mammalian tissue with a compound of formula (III):

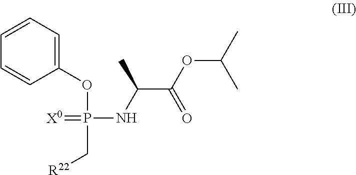

or a pharmaceutically acceptable thereof, wherein,

R²² is a monovalent substituent group of a chemotherapeutic agent, or is a monovalent substituent group of a diagnostic agent; and

X^{O} is O or absent.
[63.] The method of embodiment [62], wherein the contacting occurs in vitro.
[64.] The method of embodiment [62], wherein the contacting occurs in vivo.
[65.] The method of any one of embodiments [62]-[64], wherein the mammalian tissue is human tissue.
[66.] The method of any one of embodiments [62]-[65], wherein X^{O} is O.
[67.] The method of any one of embodiments [62]-[65], wherein X^{O} is absent.
[68.] The method of any one of embodiments [62]-[67], wherein R²² is a monovalent substituent group of a chemotherapeutic agent.
[69.] The method of any one of embodiments [62]-[67], wherein R²² is a monovalent substituent group of a diagnostic agent.
[70.] The method of any one of embodiments [62]-[69], wherein the chemotherapeutic agent is an antimetabolite selected form the group consisting of:

Capecitabine, pentyl [1-(3,4-dihydroxy-5-methyltetrahydrofuran-2-yl)-5-fluoro-2-oxo-1H-pyrimidin-4-yl]carbamate

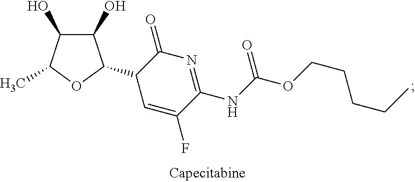 [SMILES: CCCCCOC(=O)NC1=NC(=O)C([C@@H]2O[C@H](C)[C@@H](O)[C@H]2O)C=C1F]

Gemcitabine, 4-amino-1-(2-deoxy-2,2-difluoro-β-D-erythro-pentofuranosyl)pyrimidin-2(1H)-on

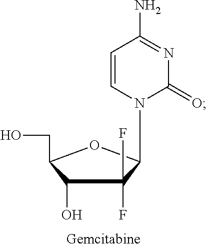 [SMILES: Nc1ccn([C@@H]2O[C@H](CO)[C@H](O)C2(F)F)c(=O)n1]

Cytarabine, 4-amino-1-[(2R,3S,4R,5R)-3,4-dihydroxy-5-(hydroxymethyl)oxolan-2-yl]pyrimidin-2-one

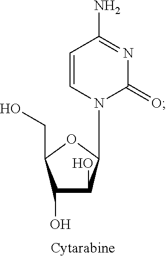

Pentostatin, (R)-3-((2R,4S,5R)-4-hydroxy-5-(hydroxymethyl)tetrahydrofuran-2-yl)-3,6,7,8-tetrahydroimidazo[4,5-d][1,3]diazepin-8-ol

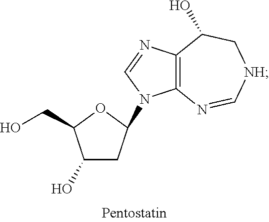 [SMILES: OC[C@H]1O[C@@H](n2cnc3c2N=CNC[C@H]3O)C[C@@H]1O]

Fludarabine, [(2R,3R,4S,5R)-5-(6-amino-2-fluoro-purin-9-yl)-3,4-dihydroxy-oxolan-2-yl]hydroxyl methyl

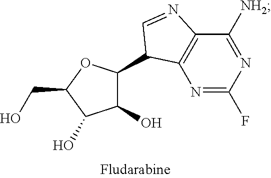 [SMILES: Nc1nc(F)nc2c1N=CC2[C@@H]1O[C@H](CO)[C@@H](O)[C@@H]1O]

Cladribine, 5-(6-amino-2-chloro-purin-9-yl)-2-(hydroxymethyl)oxolan-3-ol

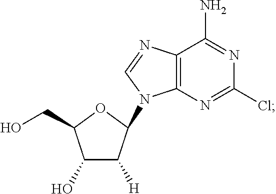

Clofarabine, 5-(6-amino-2-chloro-purin-9-yl) -4-fluoro-2-(hydroxymethyl)oxolan-3-ol;

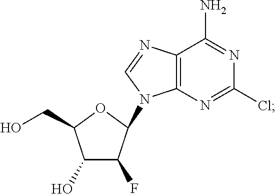 [SMILES: Nc1nc(Cl)nc2c1ncn2[C@@H]1O[C@H](CO)[C@@H](O)[C@@H]1F]

Methotrexate, (2S)-2-[(4-{[(2,4-diaminopteridin-6-yl)methyl](methyl)amino}benzoyl)amino]pentanedioic acid;

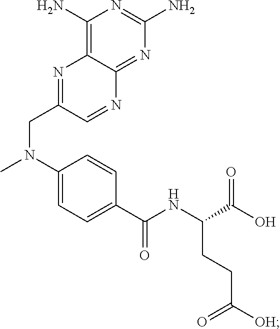 [SMILES: CN(Cc1cnc2nc(N)nc(N)c2n1)c1ccc(C(=O)N[C@@H](CCC(=O)O)C(=O)O)cc1]

Decitabine, 4-Amino-1-(2-deoxy-β-D-erythro-pentofuranosyl)-1,3,5-triazin-2(1H)-one;

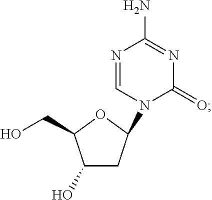 [SMILES: Nc1ncn([C@H]2C[C@H](O)[C@@H](CO)O2)c(=O)n1]

Epirubicin, (8R,10S)-10-((2S,4S,5R,6S)-4-amino-5-hydroxy-6-methyltetrahydro-2H-pyran-2-yl)-6,8,11-trihydroxy-8-(2-hydroxyacetyl)-1-methoxy-7,8,9,10-tetrahydrotetracene-5,12-dione;

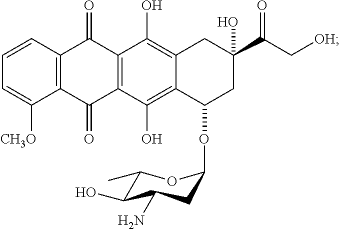

Raloxifene, [6-hydroxy-2-(4-hydroxyphenyl)-benzothiophen-3-yl]-[4-[2-(1-piperidyl)ethoxy]phenyl]-methanone;

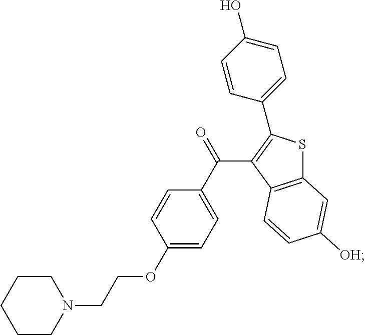 [SMILES: O=C(c1ccc(OCCN2CCCCC2)cc1)c1c(-c2ccc(O)cc2)sc2cc(O)ccc12]

Azacitidine, 4-amino-1-β-D-ribofuranosyl-1,3,5-triazin-2(1H)-one;

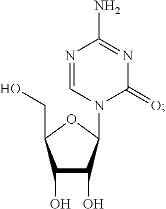

and

Ribavirin, 1-[(2R,3R,4S,5R)-3,4-dihydroxy-5-(hydroxymethyl)oxolan-2-yl]-1H-1,2,4-triazole-3-carboxamide;

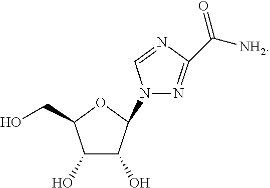 [SMILES: NC(=O)c1ncn([C@@H]2O[C@H](CO)[C@@H](O)[C@H]2O)n1]

[71.] The method of any one of embodiments [62]-[70], wherein the monovalent substituent group of the chemotherapeutic agent is selected form the group consisting of:

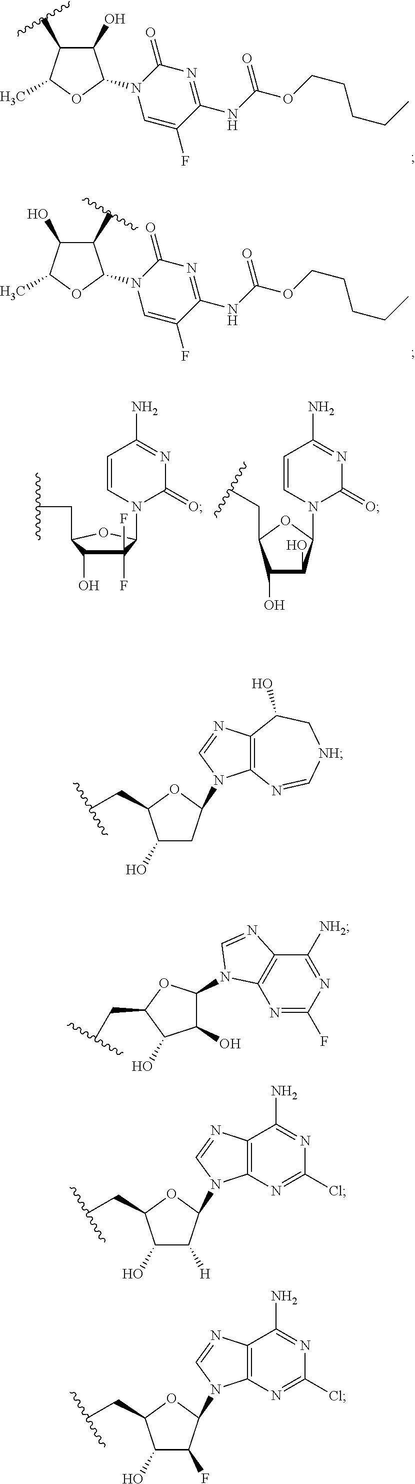

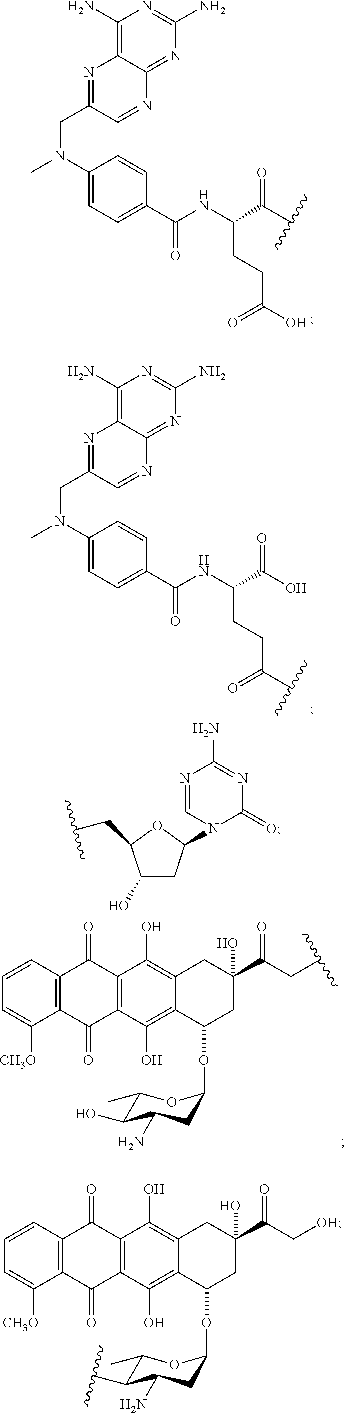

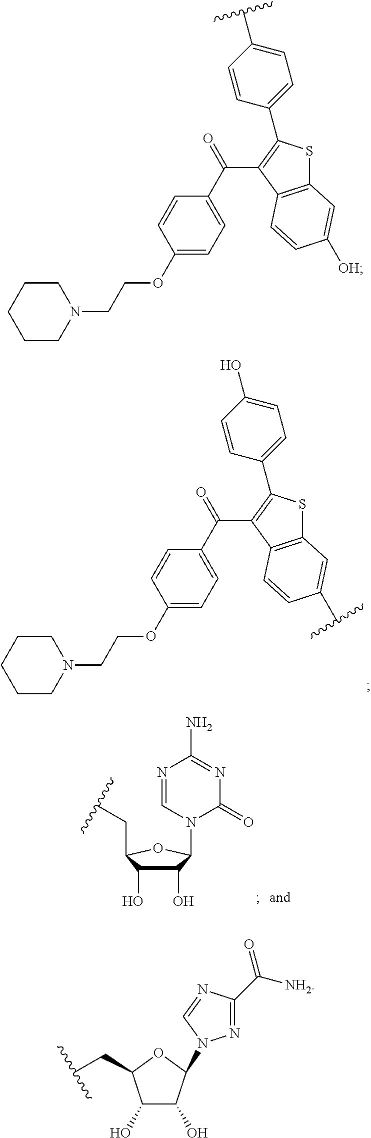

[72.] The method of any one of embodiments [62]-[71], wherein the method for targeting the tumor or cancer cell includes imaging or diagnosing the tumor or cancer cell.
[73.] The method of any one of embodiments [62]-[71], wherein the method for targeting the tumor or cancer cell includes treating the tumor or cancer cell.
[74.] A method of treating liver disease selected from liver cancer and hepatitis, the method comprising:

administering an effective amount of a compound of formula (III):

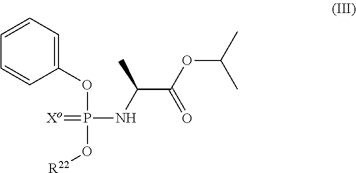

or a pharmaceutically acceptable thereof, wherein,

R²² is a monovalent substituent group of a chemotherapeutic agent, or is a monovalent substituent group of an antiviral agent; and

X^{O} is O or absent;

to a subject in need of such treatment, in a dosage and for a time effective to treat the liver disease;

wherein the compound of formula (III) is not sofosbuvir or PSI-7851.
[75.] The method of embodiment [74] wherein R²² is a chemotherapeutic agent selected from FUDR and doxorubicin, or is an antiviral agent and is ribavirin.
[76.] The method of embodiment [74] wherein the liver disease is liver cancer or hepatitis C and the compound of formula (III) is a compound of formula (Ic):

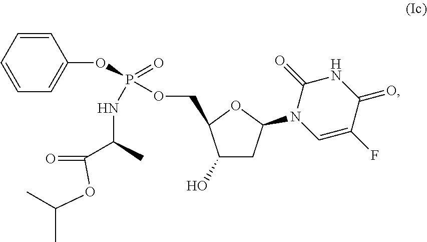 [SMILES: CC(C)OC(=O)[C@H](C)N[P@](=O)(OC[C@H]1O[C@@H](n2cc(F)c(=O)[nH]c2=O)C[C@@H]1O)Oc1ccccc1]

or a pharmaceutically acceptable salt thereof.
[77.] The method of embodiment [74] wherein R²² is capecitabine, gemcitabine, cytarabine, pentostatin, fludarabine, clabradine, clofarabine, methotrexate, decitabine, epirubicin, raloxifene, azacitidine, or ribavirin.
[78.] The method of embodiment [74], [75], or [77], wherein X^{O} is O.
[79.] The method of embodiment [74] wherein the compound of formula (III) is (IIa):

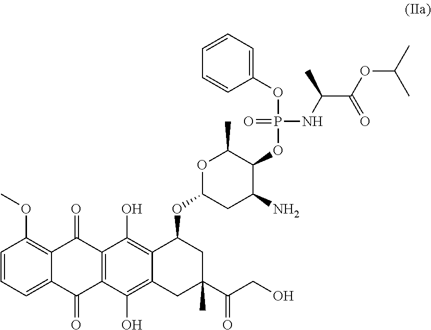 [SMILES: COc1cccc2c1C(=O)c1c(O)c3c(c(O)c1C2=O)C[C@@](C)(C(=O)CO)C[C@@H]3O[C@H]1C[C@H](N)[C@H](OP(=O)(N[C@@H](C)C(=O)OC(C)C)Oc2ccccc2)[C@H](C)O1]

or a pharmaceutically acceptable salt thereof.
[80.] The method of embodiment [74] wherein the liver disease is hepatitis C and the compound of formula (III) is a compound of formula (IIIa)

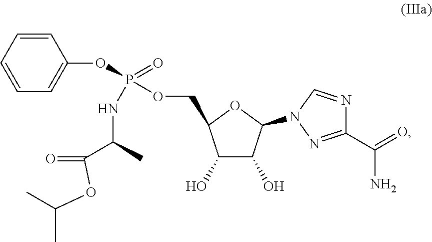 [SMILES: CC(C)OC(=O)[C@H](C)N[P@](=O)(OC[C@H]1O[C@@H](n2cnc(C(N)=O)n2)[C@H](O)[C@@H]1O)Oc1ccccc1]

or a pharmaceutically acceptable salt thereof.
[81.] The method of any one of embodiments [74]-[80] wherein the compound of formula (III) is administered in a pharmaceutical composition comprising the compound of formula (III) in combination with a pharmaceutically acceptable carrier.
[82.] The method of any one of embodiments [74]-[80] wherein the compound of formula (III) is administered to the subject orally.
[83.] The method of embodiment [76] wherein the liver disease is liver cancer and the compound of formula (Ic) is administered to the subject orally.
[84.] The method of embodiment [76] or [83] further comprising administering dexamethasone to the subject need of such treatment, in a dosage and for a time effective to treat the liver cancer.
[85.] The method of embodiment [76] or [83] wherein the compound of formula (Ic) is administered in a dosage of 0.2 to 100 mg/kg/day.
[86.] The method of embodiment [76] or [83] wherein the compound of formula (Ic) is administered in a dosage of 0.2 mg/kg/day or more.
[87.] The method of embodiment [76] or [83] wherein the compound of formula (Ic) is administered in a dosage of 1 mg/kg/day or more.
[88.] The method of embodiment [76] or [83] wherein the compound of formula (Ic) is administered in a dosage of 0.2 to 20 mg/kg/day.
[89.] The method of any one of embodiments [74]-[79] or [83], wherein the liver disease is liver cancer.
[90.] The method of embodiment [89] wherein the liver disease is hepatocellular carcinoma.
[91.] The method of embodiment [89] wherein the liver cancer is a cancer that metastasized to the liver.
[92.] The method of embodiment [91] wherein the liver cancer is colorectal carcinoma that metastasized to the liver.
[93.] The method of embodiment [74]-[78] or [80] wherein the liver disease is hepatitis.
[94.] The method of embodiment [93] wherein the liver disease is hepatitis C.
[95.] The method of embodiment [80] wherein the liver disease is hepatitis C.
[96.] A method of treating cancer, the method comprising:

administering an effective amount of a compound of formula (III):

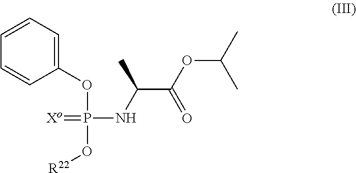

or a pharmaceutically acceptable thereof, wherein,

R²² is a monovalent substituent group of a chemotherapeutic agent, or is a monovalent substituent group of an antiviral agent; and

X^{O} is O or absent;

to a subject in need of such treatment, in a dosage and for a time effective to treat the liver disease;

wherein the compound of formula (III) is not sofosbuvir or PSI-7851.
[97.] The method of embodiment [96] wherein the compound of formula (III) is the compound of formula (Ic).
[98.] The method of embodiment [96] or [97] wherein the cancer is gastric cancer.

### EXAMPLE

### Anticancer Activity of Floxphosteridine (Compound Ic) and Floxuridine

We treated three cancer cell lines with various concentrations of Floxphosteridine (compound Ic) and floxuridine for 72 hours. The structure of floxuridine is shown below:

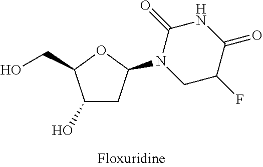 [SMILES: O=C1NC(=O)N([C@H]2C[C@H](O)[C@@H](CO)O2)CC1F]

The SK-HEP-1 and HEPG2 cell lines used in these experiments represent in vitro models for hepatic cancer. The AGS cancer cell line is an in vitro model for gastric cancer.

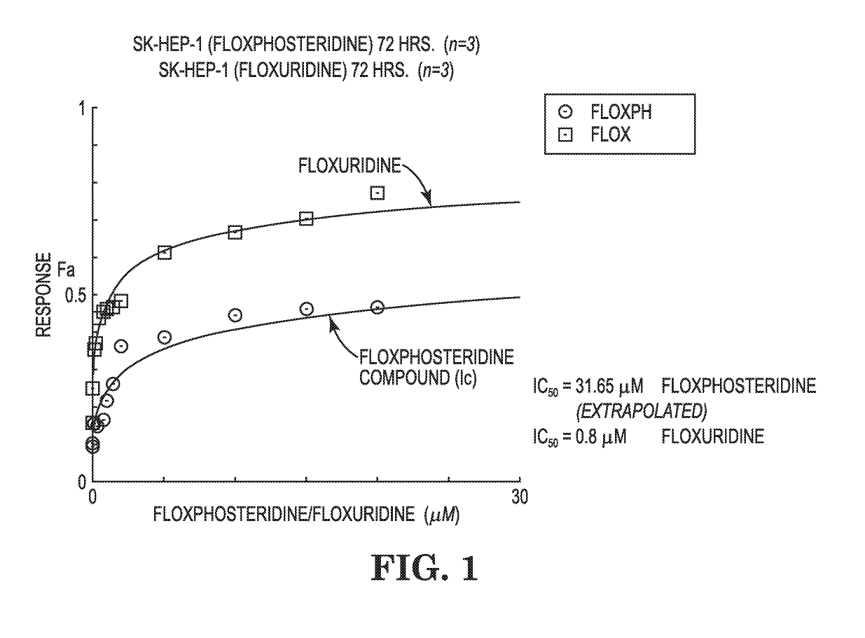

In FIG. 1, SK-HEP-1 hepatic cancer cells were treated with various concentrations (0.01-20 μM) of floxphosteridine and floxuridine for 72 hours, and cell viability was evaluated with the MTT assay. The data indicate that both agents significantly inhibited SK-HEP-1 cancer cell viability with increasing treatment concentrations. The IC50 values for floxphosteridine and floxuridine were calculated with Compusyn software to be 31.65 μM and 0.8 μM, respectively.

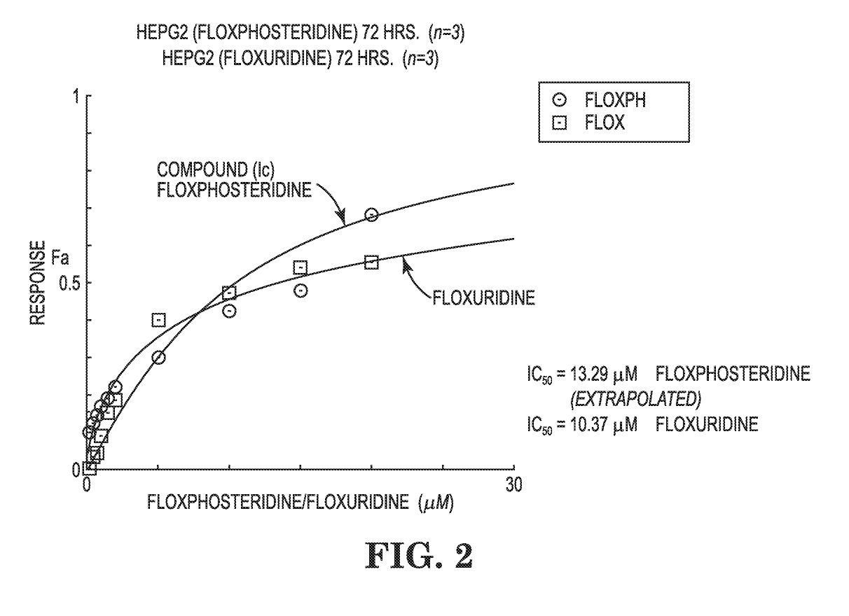

In FIG. 2, the two compounds were tested for anticancer activity against HEPG2 hepatic cancer cells. The cells were treated with various concentrations (1.0-20 μM) of floxphosteridine and floxuridine for 72 hours, and cell viability was evaluated with the MTT assay. The data indicate that both agents significantly inhibited HEPG2 cancer cell viability with increasing treatment concentrations. The IC50 values for floxphosteridine and floxuridine against HEPG2 were calculated with Compusyn software to be 13.29 μM and 10.37 μM, respectively.

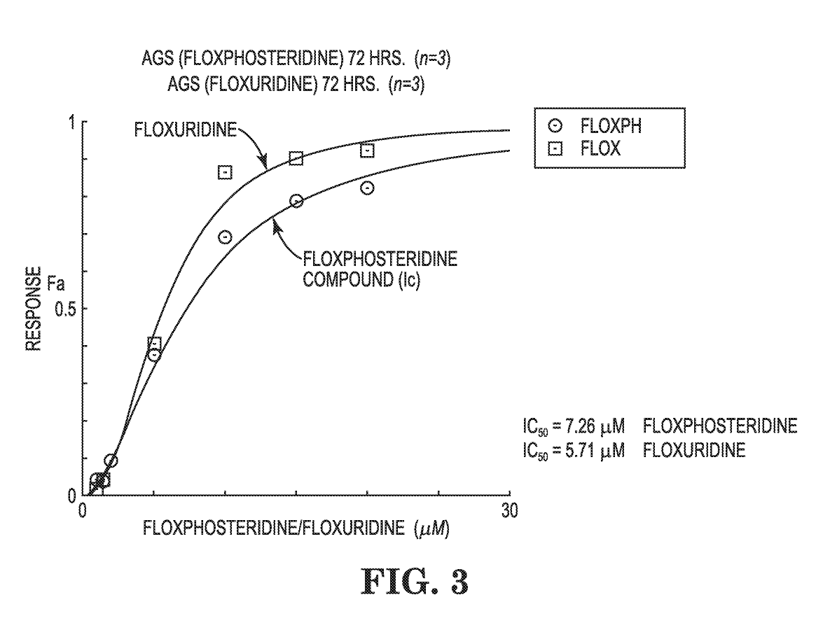

In FIG. 3, the two compounds were tested for anticancer activity against AGS gastric cancer cells. The cells were treated with various concentrations (1.0-20 μM) of floxphosteridine and floxuridine for 72 hours, and cell viability was evaluated with the MTT assay. The data indicate that both agents significantly inhibited AGS cancer cell viability with increasing treatment concentrations. The IC50 values for floxphosteridine and floxuridine against AGS cells were calculated with Compusyn software to be 7.26 μM and 5.71 μM, respectively.

This in vitro data shows that both floxphosteridine and floxuridine exhibit significant anti-cancer activity in hepatic and gastric cancer cells.

What is claimed is:

1. A method of treating liver disease selected from liver cancer and hepatitis, the method comprising:
administering an effective amount of a compound of formula (III): 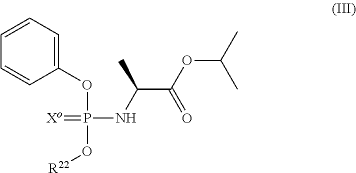
or a pharmaceutically acceptable thereof, wherein,
R²² is a monovalent substituent group of a chemotherapeutic agent, or is a monovalent substituent group of an antiviral agent; and
X^{O} is O or absent;
to a subject in need of such treatment, in a dosage and for a time effective to treat the liver disease;
wherein the compound of formula (III) is not sofosbuvir or PSI-7851.

2. The method of claim 1 wherein R²² is a chemotherapeutic agent selected from FUDR and doxorubicin, or is an antiviral agent and is ribavirin.

3. The method of claim 1 wherein the liver disease is liver cancer and the compound of formula (III) is a compound of formula (Ic): 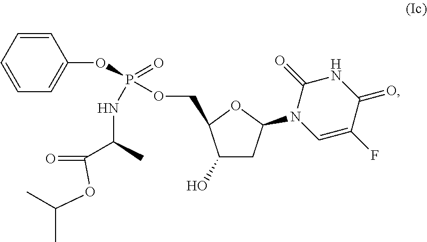 [SMILES: CC(C)OC(=O)[C@H](C)N[P@](=O)(OC[C@H]1O[C@@H](n2cc(F)c(=O)[nH]c2=O)C[C@@H]1O)Oc1ccccc1]
or a pharmaceutically acceptable salt thereof.

4. The method of claim 1 wherein R²² is capecitabine, gemcitabine, cytabarine, pentostatin, fludarabine, clabradine, clofarabine, methotrexate, decitabine, epirubicin, raloxifene, azacitidine, or ribavirin.

5. The method of claim 1 wherein X^{O} is O.

6. The method of claim 1 wherein the compound of formula (III) is (IIa): 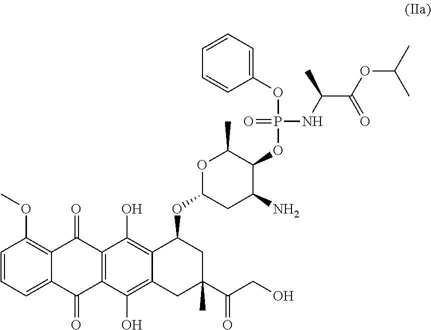 [SMILES: COc1cccc2c1C(=O)c1c(O)c3c(c(O)c1C2=O)C[C@@](C)(C(=O)CO)C[C@@H]3O[C@H]1C[C@H](N)[C@H](OP(=O)(N[C@@H](C)C(=O)OC(C)C)Oc2ccccc2)[C@H](C)O1]
or a pharmaceutically acceptable salt thereof.

7. The method of claim 1 wherein the liver disease is hepatitis and the compound of formula (III) is a compound of formula (IIIa) 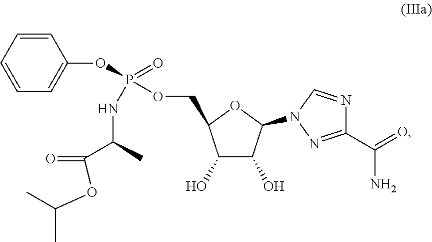 [SMILES: CC(C)OC(=O)[C@H](C)N[P@](=O)(OC[C@H]1O[C@@H](n2cnc(C(N)=O)n2)[C@H](O)[C@@H]1O)Oc1ccccc1]
or a pharmaceutically acceptable salt thereof.

8. The method of claim 1 wherein the compound of formula (III) is administered in a pharmaceutical composition comprising the compound of formula (III) in combination with a pharmaceutically acceptable carrier.

9. The method of claim 1 wherein the compound of formula (III) is administered to the subject orally.

10. The method of claim 3 wherein the liver disease is liver cancer and the compound of formula (Ic) is administered to the subject orally.

11. The method of claim 3 wherein the liver disease is liver cancer, the method further comprising administering dexamethasone to the subject need of such treatment, in a dosage and for a time effective to treat the liver cancer.

12. The method of claim 3 wherein the compound of formula (Ic) is administered in a dosage of 0.2 to 100 mg/kg/day.

13. The method of claim 3 wherein the compound of formula (Ic) is administered in a dosage of 0.2 mg/kg/day or more.

14. The method of claim 3 wherein the compound of formula (Ic) is administered in a dosage of 1 mg/kg/day or more.

15. The method of claim 3 wherein the compound of formula (Ic) is administered in a dosage of 0.2 to 20 mg/kg/day.

16. The method of claim 1 wherein the liver disease is liver cancer.

17. The method of claim 16 wherein the liver disease is hepatocellular carcinoma.

18. The method of claim 16 wherein the liver cancer is a cancer that metastasized to the liver.

19. The method of claim 18 wherein the liver cancer is colorectal carcinoma that metastasized to the liver.

20. The method of claim 1 wherein the liver disease is hepatitis.

21. The method of claim 20 wherein the liver disease is hepatitis C.

22. The method of claim 7 wherein the liver disease is hepatitis C.

23. A method of treating gastric cancer, the method comprising:
administering an effective amount of a compound of formula (III): 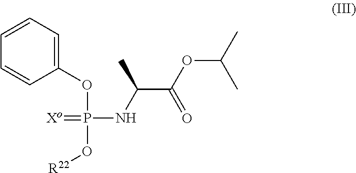
or a pharmaceutically acceptable thereof, wherein,
R²² is a monovalent substituent group of a chemotherapeutic agent, or is a monovalent substituent group of an antiviral agent; and
X^{O} is O or absent;
to a subject in need of such treatment, in a dosage and for a time effective to treat the liver disease;
wherein the compound of formula (III) is not sofosbuvir or PSI-7851.

24. The method of claim 23 wherein the compound is the compound of formula (Ic): 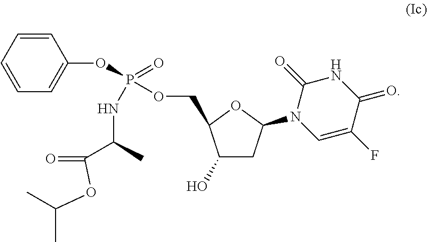 [SMILES: CC(C)OC(=O)[C@H](C)N[P@](=O)(OC[C@H]1O[C@@H](n2cc(F)c(=O)[nH]c2=O)C[C@@H]1O)Oc1ccccc1]
